(19) United States
(12) Patent Application Publication
(10) Pub. No.: US 2024/0390623 A1
(43) Pub. Date: Nov. 28, 2024

(54) FACE MASK

(71) Applicant: HSINER CO., LTD., Taichung City (TW)

(72) Inventor: Eric CHANG, Taichung City (TW)

(21) Appl. No.: 18/352,560

(22) Filed: Jul. 14, 2023

(30) Foreign Application Priority Data
May 26, 2023 (TW) 112119772

(51) Int. Cl. A61M 16/06

(52) U.S. Cl. CPC A61M 16/0605

(57) ABSTRACT

A face mask includes a main body and a soft cover. The main body is made of a rigid plastic material, and includes a peripheral edge that has a bottom section, a top section that is opposite to the bottom section and that is curved, and two side sections. The soft cover is made of a flexible plastic material, and is fixed to the peripheral edge of the main body. The soft cover has a lower section fixed to the bottom section of the peripheral edge, a nose section opposite to the lower section, and two side sections respectively corresponding to the side sections of the peripheral edge. The nose section has a first deformation region, and a second deformation region connected to the first deformation region, protruding relative to the side sections of the soft cover, and sealingly connected to the top section of the peripheral edge.

### CROSS-REFERENCE TO RELATED APPLICATION

This application claims priority to Taiwanese Invention patent application Ser. No. 11/211,9772, filed on May 26, 2023.

### FIELD

The disclosure relates to a medical device, more particularly to a face mask adaptable for different nasal bridges.

### BACKGROUND

A conventional face mask includes a mask body, an assembly, and a cover body that is connected to the mask body through the assembly.

The mask body is made of a soft material, and is flexible and deformable. However, since heights of nasal bridges of different users of the face mask vary and deformation of the cover body is restricted by the structure thereof when the face mask is being worn, the cover body may not fit some users.

### SUMMARY

Therefore, an object of the disclosure is to provide a face mask adaptable for different nasal bridges that can alleviate at least one of the drawbacks of the prior art.

According to the disclosure, the face mask includes a main body and a soft cover. The main body is made of a rigid plastic material, is cup-shaped, and includes a peripheral edge, and a protrusion that is spaced apart from the peripheral edge in a transverse direction. The main body is tapered from the peripheral edge toward the protrusion. The soft cover is made of a flexible plastic material, surrounds the main body, and is fixed to the peripheral edge of the main body.

The peripheral edge of the main body has a bottom section, a top section that is opposite to the bottom section, and two side sections that are connected between the bottom section and the top section. The top section is curved, and has two end portions that are respectively connected to the side sections, a central portion that is located between the end portions and the protrusion, and two oblique portions each of which is connected between a respective one of the end portions and the central portion.

The soft cover has a lower section that is fixed to the bottom section of the peripheral edge, a nose section that is opposite to the lower section, and two side sections that are connected between the lower section and the nose section and that respectively correspond to the side sections of the peripheral edge. The nose section has a first deformation region, and a second deformation region that is connected to the first deformation region and that protrudes relative to the side sections of the soft cover. The second deformation region is sealingly connected to the top section of the peripheral edge, and has a wide edge that is connected to the first deformation region, a narrow edge that is connected to the central portion of the peripheral edge, and two inclined edges that are connected between the wide edge and the narrow edge.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a perspective view illustrating an embodiment of a face mask adaptable for different nasal bridges according to the disclosure.

FIG. 2 is a fragmentary enlarged perspective view of the embodiment.

FIG. 3 is a sectional view of the embodiment.

FIG. 4 is a fragmentary enlarged sectional view of FIG. 3.

FIG. 5 is a front view of the embodiment.

FIG. 6 is a sectional view similar to FIG. 3, illustrating a first deformation region of a soft cover that is compressed by a normal nasal bridge and that is deformed.

FIG. 7 is a sectional view similar to FIG. 6, illustrating a second deformation region of the soft cover that is compressed by a higher nasal bridge and that is deformed.

### DETAILED DESCRIPTION

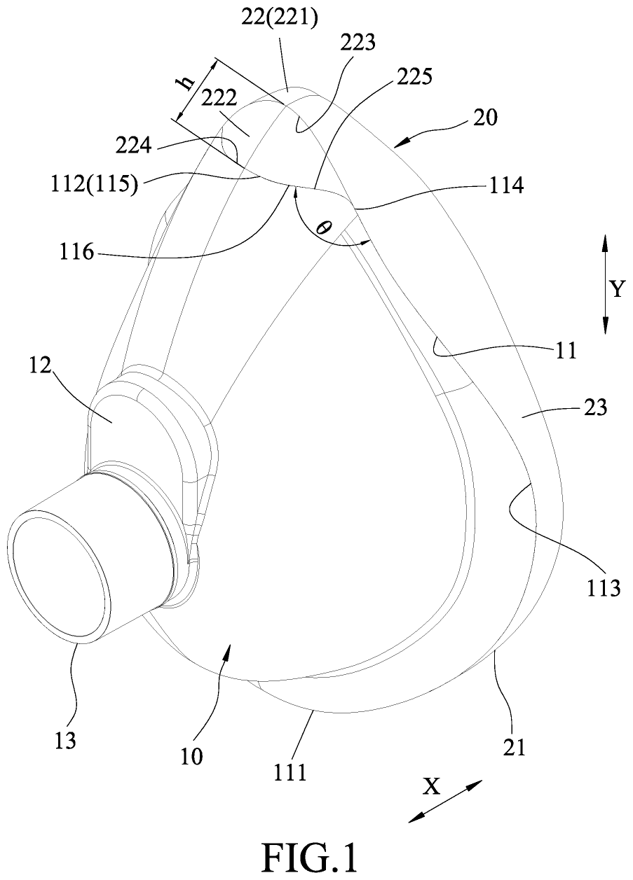

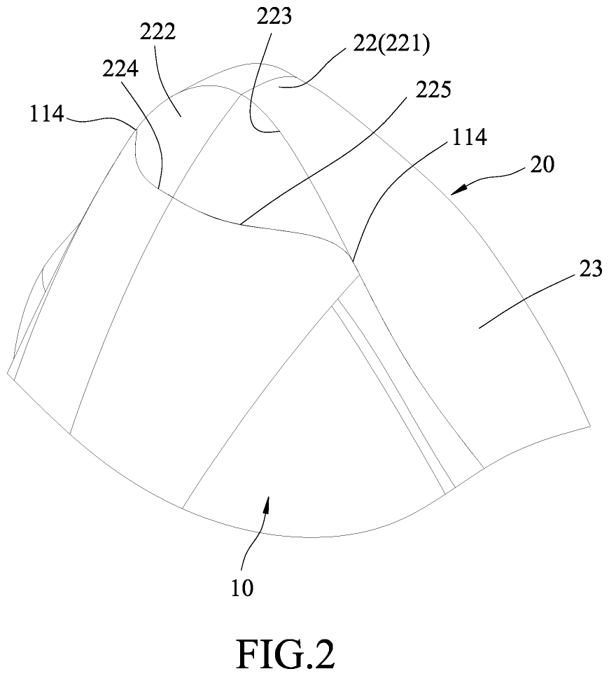

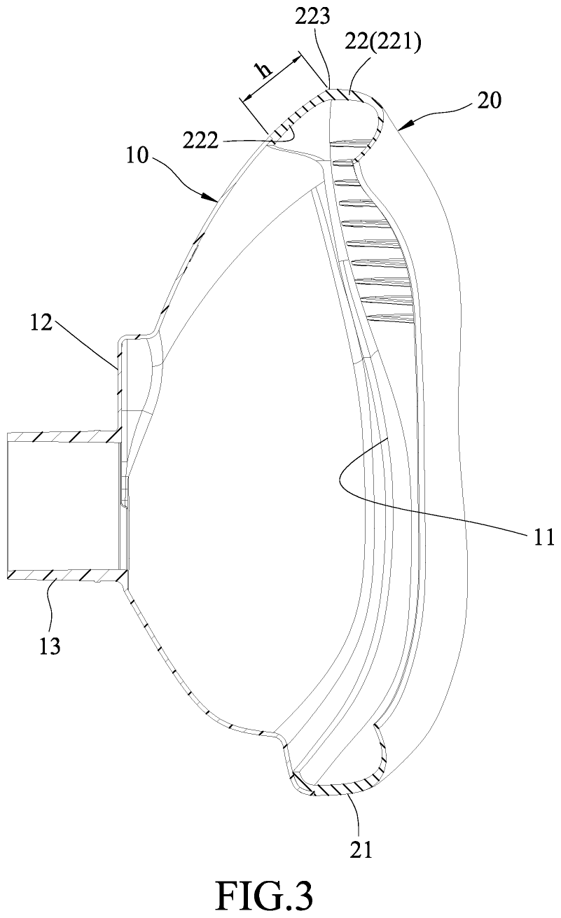

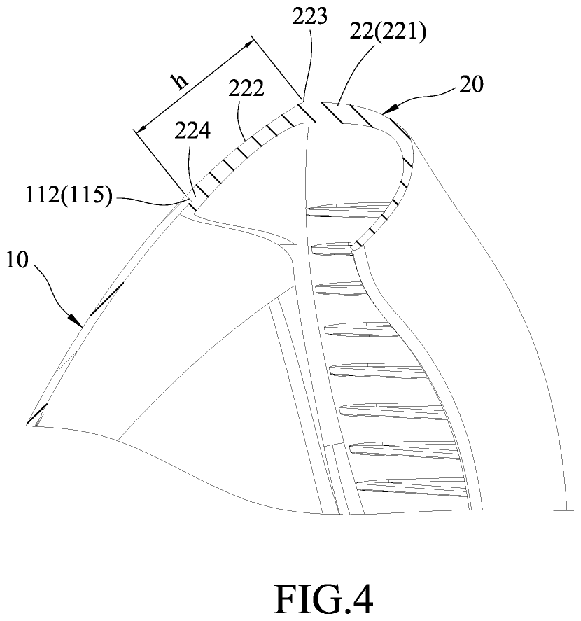

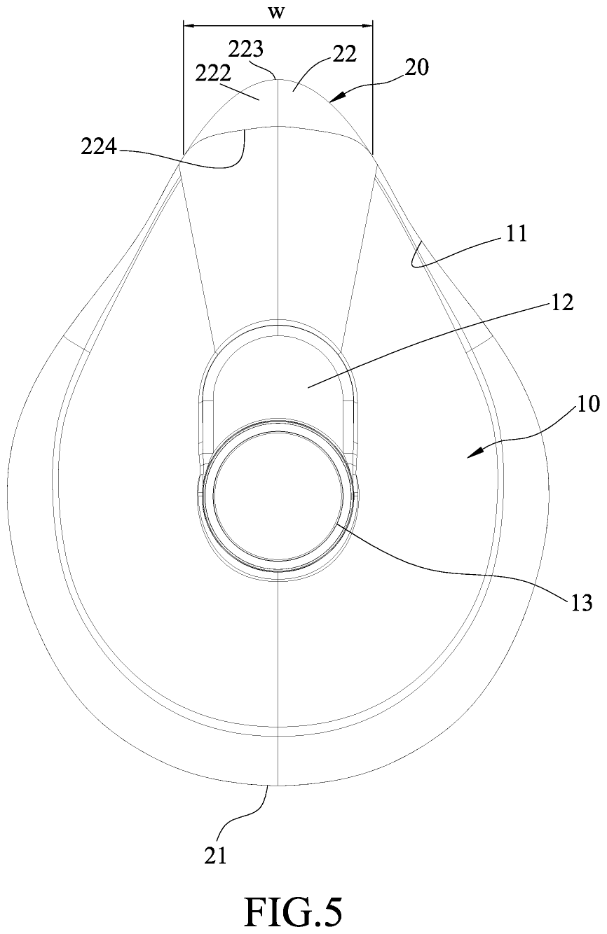

Referring to FIGS. 1 to 5, according to the disclosure, an embodiment of a face mask adaptable for different nasal bridges includes a main body 10 and a soft cover 20.

The main body 10 is made of a rigid plastic material, is cup-shaped, and includes a peripheral edge 11, a protrusion 12 that is spaced apart from the peripheral edge 11 in a transverse direction (X), and a connecting pipe 13 that is fixed to the protrusion 12 and that extends in the transverse direction (X). The main body 10 is tapered from the peripheral edge 11 toward the protrusion 12. The peripheral edge 11 has a bottom section 111, a top section 112 that is opposite to the bottom section 111 in a longitudinal direction (Y) perpendicular to the transverse direction (X), and two side sections 113 that are connected between the bottom section 111 and the top section 112. The top section 112 is curved, and has two end portions 114 that are respectively connected to the side sections 113, a central portion 115 that is located between the end portions 114 and the protrusion 12, and two oblique portions 116 each of which is connected between a respective one of the end portions 114 and the central portion 115. A ratio of a distance (w) between the end portions 114 in a direction perpendicular to the transverse direction (X) to a total length of the peripheral edge 11 ranges from 0.09 to 0.11. Each of the oblique portions 116 cooperates with a respective one of the side sections 113 that is connected to the respective one of the end portions 114 to form an angle (0) that is an obtuse angle.

The soft cover 20 is made of a flexible plastic material, surrounds the main body 10, and is fixed to the peripheral edge 11 of the main body 10 by using overmolding technology. The soft cover 20 has a lower section 21 that is fixed to the bottom section 111 of the peripheral edge 11, a nose section 22 that is opposite to the lower section 21, and two side sections 23 that are connected between the lower section 21 and the nose section 22 and that respectively correspond to the side sections 113 of the peripheral edge 11. The nose section 22 has a first deformation region 221, and a second deformation region 222 that is connected to the first deformation region 221 and that protrudes relative to the side sections 23 of the soft cover 20. The second deformation region 222 is sealingly connected to the top section 112 of the peripheral edge 11, and has a wide edge 223 that is connected to the first deformation region 221, a narrow edge 224 that is connected to the central portion 115 of the peripheral edge 11, and two inclined edges 225 that are connected between the wide edge 223 and the narrow edge 224. A ratio of a distance (h) between the wide edge 223 (in particular, a central point of the wide edge 223) and the narrow edge 224 (in particular, a central point of the narrow edge 224) of the second deformation region 222 of the nose section 22 to the distance (w) between the end portions 114 of the top section 112 of the peripheral edge 11 in a direction perpendicular to the transverse direction (X) ranges from 0.3 to 0.35. A cross section of the first deformation region 221 of the nose section 22 that is perpendicular to a direction perpendicular to the transverse direction (X) and the longitudinal direction (Y) is invertedly J-shaped. A cross section of the lower section 21 that is perpendicular to the direction perpendicular to the transverse direction (X) and the longitudinal direction (Y) is invertedly J-shaped. Cross sections of the peripheral sections 23 that are perpendicular to the longitudinal direction (Y) are invertedly J-shaped.

To further understand generated functions, used technical means, and the expected effect of the disclosure, the disclosure is further described below.

As shown in FIGS. 1 to 5, when the face mask of the disclosure is not being worn, the soft cover 20 is smoothly connected to the main body 10 due to not being affected by an external force.

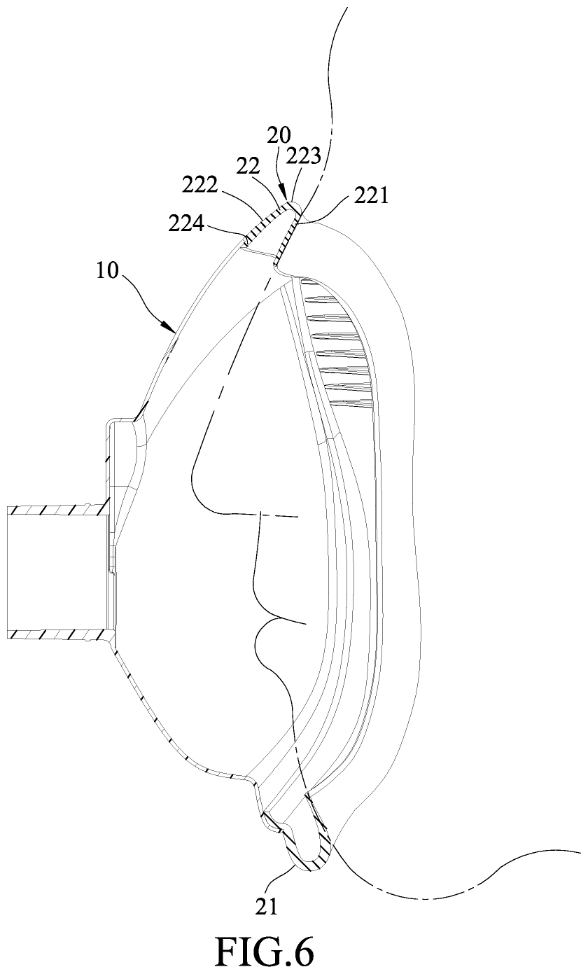

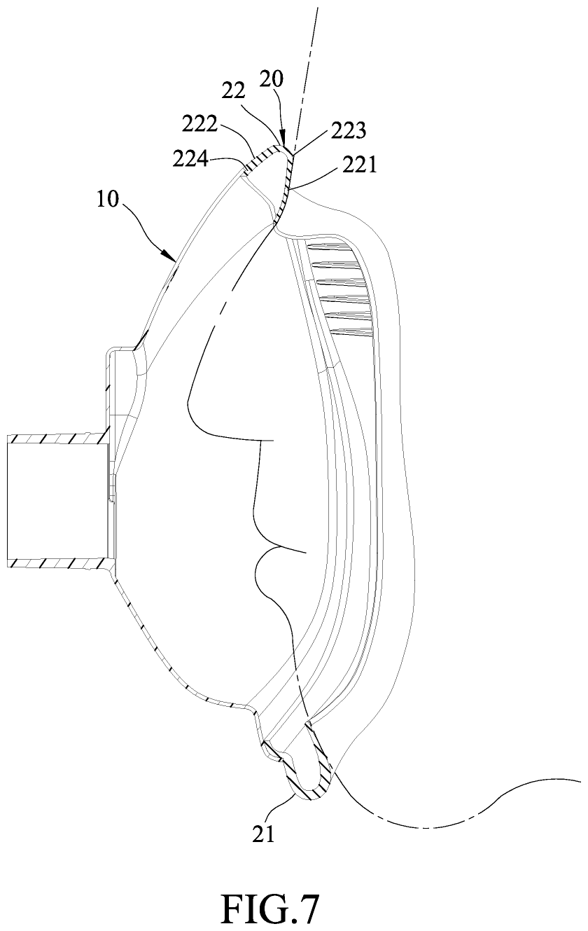

Referring to FIGS. 6 and 7, in use, when the face mask is being worn, the top section 112 of the peripheral edge 11 of the main body 10 and the second deformation region 222 of the nose section 22 of the soft cover 20 positionally correspond to the nasal bridge of the user, and the bottom section 111 of the peripheral edge 11 of the main body 10 and the lower section 21 of the soft cover 20 positionally correspond to the chin of the user. Meanwhile, the nose section 22 of the soft cover 20 is abutted on the nasal bridge of the user, and the lower section 21 of the soft cover 20 is abutted on the chin of the user.

As shown in FIG. 6, when the face mask is being worn by a user with a normal nasal bridge, the first deformation region 221 of the soft cover 20 is compressed by the nasal bridge of the user and is deformed, but the second deformation region 222 is kept smooth (that is to say, the second deformation region 222 is not compressed and deformed).

As shown in FIG. 7, when the face mask is being worn by another user with a higher nasal bridge, the second deformation region 222 is compressed by the higher nasal bridge and is deformed inwardly, so that a cross section of the second deformation region 222 that is perpendicular to the direction perpendicular to the transverse direction (X) and the longitudinal direction (Y) is invertedly U-shaped, and a distance between the wide edge 223 and the narrow edge 224 of the second deformation region 222 in the transverse direction (X) is shortened.

Since the first deformation region 221 and the second deformation region 222 of the nose section 22 of the soft cover 20 are flexible and deformable, when the nose section 22 of the soft cover 20 is compressed by nasal bridges with different heights, the first deformation region 221 or the second deformation region 222 may deform relative to the main body 10. Furthermore, resilience of the nose section 22 allows the nose section 22 to fit more closely to the nasal bridges.

In summary, according to the disclosure, the main body 10 is made of the rigid plastic material, the top section 112 of the peripheral edge 11 of the main body 10 is curved, the soft cover 20 is made of the flexible plastic material, and the nose section 22 of the soft cover 20 is sealingly connected to the top section 112 of the main body 10, so that the nose section 22 has a relatively high deformability. Furthermore, when the face mask is being worn by a user, the first deformation region 221 and the second deformation region 222 of the nose section 22 deform in different levels such that the face mask may be adaptable for nasal bridges with different heights. In addition, the face mask of the disclosure has a simple structure, and is easy to assemble. Therefore, the object of the disclosure is achieved.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features.

In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A face mask adaptable for different nasal bridges, comprising:
a main body being made of a rigid plastic material, being cup-shaped, and including a peripheral edge, and a protrusion that is spaced apart from said peripheral edge in a transverse direction, said main body being tapered from said peripheral edge toward said protrusion; and
a soft cover being made of a flexible plastic material, surrounding said main body, and being fixed to said peripheral edge of said main body;
said peripheral edge having a bottom section, a top section that is opposite to said bottom section, and two side sections that are connected between said bottom section and said top section, said top section being curved, and having two end portions that are respectively connected to said side sections, a central portion that is located between said end portions and said protrusion, and two oblique portions each of which is connected between a respective one of said end portions and said central portion,
said soft cover having a lower section that is fixed to said bottom section of said peripheral edge, a nose section that is opposite to said lower section, and two side sections that are connected between said lower section and said nose section and that respectively correspond to said side sections of said peripheral edge, said nose section having a first deformation region, and a second deformation region that is connected to said first deformation region and that protrudes relative to said side sections of said soft cover, said second deformation region being sealingly connected to said top section of said peripheral edge, and having a wide edge that is connected to said first deformation region, a narrow edge that is connected to said central portion of said peripheral edge, and two inclined edges that are connected between said wide edge and said narrow edge.

2. The face mask as claimed in claim 1, wherein a ratio of a distance between said end portions of said top section of said peripheral edge to a total length of said peripheral edge ranges from 0.09 to 0.11.

3. The face mask as claimed in claim 2, wherein a ratio of a distance between said wide edge and said narrow edge of said second deformation region to the distance between said end portions of said top section of said peripheral edge ranges from 0.3 to 0.35.

4. The face mask as claimed in claim 1, wherein each of said oblique portions of said top section of said peripheral edge cooperates with a respective one of said side sections of said peripheral edge that is connected to the respective one of said end portions to form an obtuse angle.

5. The face mask as claimed in claim 1, wherein said main body further includes a connecting pipe that is fixed to said protrusion.

6. The face mask as claimed in claim 1, wherein said bottom section and said top section of said peripheral edge of said main body are opposite to each other in a longitudinal direction that is perpendicular to the transverse direction, a cross section of said first deformation region of said nose section of said soft cover that is perpendicular to a direction perpendicular to the transverse direction and the longitudinal direction being invertedly J-shaped.

7. The face mask as claimed in claim 6, wherein a cross section of said lower section of said soft cover that is perpendicular to the direction perpendicular to the transverse direction and the longitudinal direction is invertedly J-shaped, cross sections of said peripheral sections that are perpendicular to the longitudinal direction being invertedly J-shaped.
